(19) United States
(12) Patent Application Publication
(10) Pub. No.: US 2025/0041086 A1
(43) Pub. Date: Feb. 6, 2025

(54) DEVICES, SYSTEMS, AND METHODS FOR DEPLOYING AND RECAPTURING A MEDICAL DEVICE

(71) Applicant: BOSTON SCIENTIFIC SCIMED, INC., MAPLE GROVE, MN (US)

(72) Inventors: James M. Anderson, Corcoran, MN (US); Daniel Presteng, Corcoran, MN (US)

(73) Assignee: BOSTON SCIENTIFIC SCIMED, INC., MAPLE GROVE, MN (US)

(21) Appl. No.: 18/792,929

(22) Filed: Aug. 2, 2024

Related U.S. Application Data
(60) Provisional application No. 63/530,727, filed on Aug. 4, 2023.

(51) Int. Cl. A61F 2/95; A61F 2/24

(52) U.S. Cl. CPC A61F 2/95; A61F 2/2436; A61F 2002/9528; A61F 2002/9534

(57) ABSTRACT

A device for aiding in recapturing a medical device may include an elongate shaft and an expandable sheath extension coupled to a distal end of the elongate shaft. The expandable sheath extension may be laterally offset from the elongate shaft and may be self-biased toward a radially collapsed configuration. A system for deploying and recapturing a medical device may include a deployment catheter including an elongate tubular member having a lumen configured to slidably receive a medical device, and a device for aiding in recapturing the medical device after the medical device has been deployed. The elongate shaft may be configured to slidably advance the expandable sheath extension through the lumen to a guiding position wherein a proximal end of the expandable sheath extension is disposed within the lumen and a distal end of the expandable sheath extension is disposed distal of the elongate tubular member.

### CROSS-REFERENCE TO RELATED APPLICATIONS

This application claims the benefit of priority of U.S. Provisional Application No. 63/530,727 filed Aug. 4, 2023, the entire disclosure of which is hereby incorporated by reference.

### TECHNICAL FIELD

The disclosure relates generally to devices, systems, and methods for deploying and/or recapturing medical devices.

### BACKGROUND

In some instances, performing percutaneous medical procedures may require the insertion and/or maneuvering of expandable medical devices through a patient's vasculature. However, inserting the medical device into the vasculature may result in undesirable forces being applied to the vessel walls. Additionally, the expandable medical device may need to be recaptured, repositioned, and/or removed after expansion. Of the known devices, systems, and methods for deploying and/or recapturing a medical device, each has certain advantages and disadvantages. There is an ongoing need to provide alternative devices, systems, and/or methods.

### SUMMARY

In one example, a device for aiding in recapturing a medical device may comprise an elongate shaft and an expandable sheath extension coupled to a distal end of the elongate shaft. The expandable sheath extension may be laterally offset from the elongate shaft. The expandable sheath extension may be self-biased toward a radially collapsed configuration.

In addition or alternatively to any example disclosed herein, the expandable sheath extension extends circumferentially around a central longitudinal axis extending parallel to the elongate shaft.

In addition or alternatively to any example disclosed herein, the expandable sheath extension is circumferentially discontinuous.

In addition or alternatively to any example disclosed herein, the expandable sheath extension comprises a C-shaped collar proximate a proximal end of the expandable sheath extension.

In addition or alternatively to any example disclosed herein, the C-shaped collar is fixedly attached to the elongate shaft.

In addition or alternatively to any example disclosed herein, the C-shaped collar is formed from a metallic material.

In addition or alternatively to any example disclosed herein, the expandable sheath extension comprises at least one polymeric sheet rolled into a tubular member in the radially collapsed configuration.

In addition or alternatively to any example disclosed herein, the C-shaped collar is at least partially embedded within the at least one polymeric sheet.

In addition or alternatively to any example disclosed herein, the tubular member has an outer diameter of less than 4.667 millimeters in the radially collapsed configuration.

In addition or alternatively to any example disclosed herein, application of a radially outward force against an inner surface of the expandable sheath extension causes the at least one polymeric sheet to expand radially and circumferentially.

In addition or alternatively to any example disclosed herein, a system for deploying and recapturing a medical device may comprise a deployment catheter comprising an elongate tubular member having a lumen extending therethrough, the lumen being configured to slidably receive a medical device, and a device for aiding in recapturing the medical device after the medical device has been deployed distal of the elongate tubular member. The device may comprise an elongate shaft and an expandable sheath extension coupled to a distal end of the elongate shaft. The expandable sheath extension may be laterally offset from the elongate shaft. The expandable sheath extension may be self-biased toward a radially collapsed configuration.

In addition or alternatively to any example disclosed herein, the elongate shaft is configured to slidably advance the expandable sheath extension through the lumen of the elongate tubular member to a guiding position wherein a proximal end of the expandable sheath extension is disposed within the lumen of the elongate tubular member and a distal end of the expandable sheath extension is disposed distal of the elongate tubular member.

In addition or alternatively to any example disclosed herein, in the guiding position the expandable sheath extension is configured to guide the medical device radially inward toward the lumen of the elongate tubular member as the medical device is pulled proximally.

In addition or alternatively to any example disclosed herein, the medical device has an outer extent in an unconstrained configuration that is greater than an inner diameter of the elongate tubular member at a distal end of the elongate tubular member.

In addition or alternatively to any example disclosed herein, the expandable sheath extension is configured to be translated in a lateral direction relative to an elongate member extending proximally from the medical device through the lumen of the elongate tubular member to shift the expandable sheath extension from a first position alongside the elongate member to a second position the elongate member extends longitudinally through the expandable sheath extension.

In addition or alternatively to any example disclosed herein, the elongate shaft is sized and configured to slide longitudinally alongside the elongate member within the lumen of the elongate tubular member.

In addition or alternatively to any example disclosed herein, a method of deploying and recapturing a medical device may comprise: deploying a medical device from a deployment catheter, the deployment catheter comprising an elongate tubular member having a lumen extending therethrough, wherein the lumen is configured to slidably receive the medical device disposed at a distal end of an elongate member; positioning an expandable sheath extension coupled to a distal end of an elongate shaft in a first position alongside the elongate member, wherein the expandable sheath extension is laterally offset from the elongate shaft and wherein the expandable sheath extension is self-biased toward a radially collapsed configuration; translating the expandable sheath extension from the first position in a lateral direction relative to the elongate member to a second position wherein the elongate member extends longitudinally through the expandable sheath extension; advancing the expandable sheath extension along the elongate member to a guiding position proximate a distal end of the elongate tubular member; and pulling the medical device proximally into the elongate tubular member through the expandable sheath extension.

In addition or alternatively to any example disclosed herein, at least a portion of the expandable sheath extension is configured to expand radially upon engagement with the medical device.

In addition or alternatively to any example disclosed herein, when the expandable sheath extension is disposed in the second position in the radially collapsed configuration, at least a portion of the expandable sheath extension completely surrounds the elongate tubular member.

In addition or alternatively to any example disclosed herein, after deploying the medical device from the deployment catheter, the medical device radially expands to an outer extent greater than an inner diameter of the elongate tubular member at a distal end of the elongate tubular member.

In addition or alternatively to any example disclosed herein, the expandable sheath extension is circumferentially discontinuous.

In addition or alternatively to any example disclosed herein, the expandable sheath extension circumferentially overlaps itself to form a tubular member in the radially collapsed configuration

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic view illustrating selected aspects of a deployment catheter and a medical device deployed therefrom;

FIG. 2 is a schematic view illustrating selected aspects of an attempt to recapture the medical device using the deployment catheter of FIG. 1;

FIG. 3 is a schematic view illustrating selected aspects of a device for aiding in recapturing a medical device; and

FIG. 4 is a schematic view illustrating selected aspects of the device of FIG. 3 engaging a medical device; and

FIGS. 5-8 schematically illustrate selected aspects of a method of deploying and/or recapturing a medical device using the device of FIGS. 3-4.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary clement shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

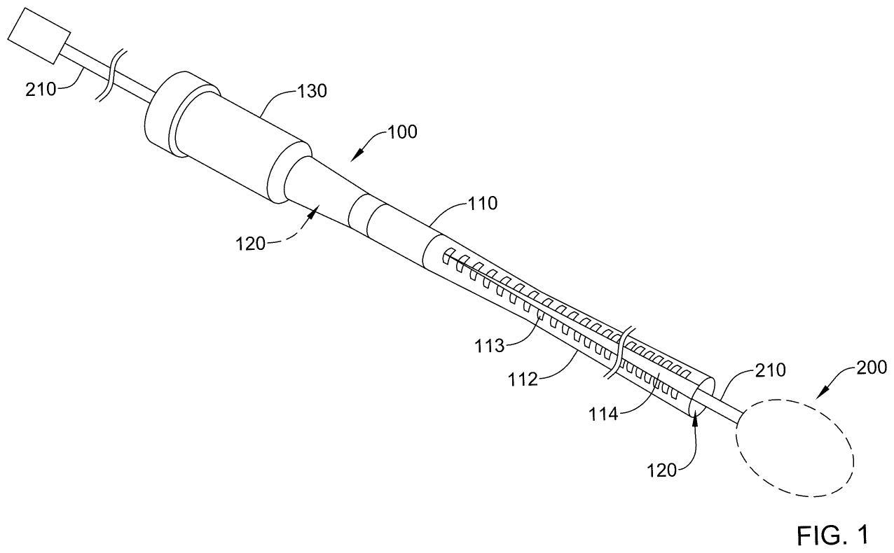

FIG. 1 illustrates selected aspects of a deployment catheter 100. In some embodiments, the term “deployment catheter” may be used interchangeably with “introducer”, and the like. In some embodiments, the deployment catheter 100 may comprise an elongate tubular member 110 having a lumen 120 extending therethrough. In some embodiments, the deployment catheter 100 may comprise a proximal hub 130 disposed at and/or coupled to a proximal end of the elongate tubular member 110. The proximal hub 130 may be in fluid communication with the lumen 120. In some embodiments, the proximal hub 130 may include a hemostatic valve or seal disposed therein.

In some embodiments, the elongate tubular member 110 may comprise an outer sheath 112 and an inner liner 114. In some embodiments, the lumen 120 may extend within and/or through the inner liner 114. In some embodiments, the outer sheath 112 may be disposed over, around, and/or about the inner liner 114. In some embodiments, the elongate tubular member 110 may include a tapered region disposed proximal to a distal region of the elongate tubular member 110. In at least some embodiments, the tapered region may taper radially inward toward the distal region and/or a distal end of the elongate tubular member 110.

The inner liner 114 may include at least one folded portion extending along at least the distal region of the elongate tubular member 110 in a delivery configuration and/or a folded configuration. In some embodiments, the inner liner 114 may extend along at least the distal region and the tapered region of the elongate tubular member 110 in a delivery configuration and/or a folded configuration. In some embodiments, the at least one folded portion may extend along an entire length of the inner liner 114 and/or the elongate tubular member 110 in the delivery configuration and/or the folded configuration. In some embodiments, the at least one folded portion may include one folded portion, two folded portions, three folded portions, four folded portions, five folded portions, or more folded portions, as desired in a particular configuration. The inner liner 114 may be disposed radially inward of the outer sheath 112. In some embodiments, each of and/or some of the at least one folded portion may fold back on itself to form, for example, a wave shape, an S-shape, a T-shape, a Z-shape, and/or combinations of these when viewed in cross-section in the delivery configuration and/or the folded configuration. Other shapes and/or configurations are also contemplated.

In the delivery configuration, the inner liner 114 may have a generally annular shape. Other shapes and/or configurations are also contemplated. In the delivery configuration and/or the folded configuration, the lumen 120 may have a first inner diameter and/or a first inner extent at the distal region and/or the distal end of the elongate tubular member 110 measured through and normal to a central longitudinal axis defined by the inner surface of the inner liner 114. The inner liner 114 may be configured to radially expand from the delivery configuration and/or the folded configuration to an expanded configuration when subjected to a radially outward force from within the lumen 120, as seen in FIG. 1.

In the expanded configuration, the lumen 120 may have a second inner diameter and/or a second inner extent at the distal region and/or the distal end of the elongate tubular member 110 measured through and normal to the central longitudinal axis that is greater than the first inner diameter and/or the first inner extent. At least one of the at least one folded portion may at least partially unfold when the inner liner 114 radially expands from the delivery configuration and/or the folded configuration toward and/or to the expanded configuration. Similarly, in the delivery configuration and/or the folded configuration, the inner liner 114 may have a first outer diameter and/or a first outer extent at the distal region and/or the distal end of the elongate tubular member 110 measured through and normal to the central longitudinal axis defined by the outer surface of the inner liner 114. In the expanded configuration, the inner liner 114 may have a second outer diameter and/or a second outer extent at the distal region and/or the distal end of the elongate tubular member 110 measured through and normal to the central longitudinal axis that is greater than the first outer diameter and/or the first outer extent.

In some embodiments, the inner liner 114 may be configured to permit the lumen 120 to radially expand from the first inner diameter and/or the first inner extent toward and/or to the second inner diameter and/or the second inner extent when subjected to the radially outward force from within the lumen 120. In some embodiments, the inner liner 114 may be configured to expand radially outward from the first inner diameter and/or the first inner extent to a predetermined second inner diameter and/or a predetermined second inner extent. For example, the second inner diameter and/or the second inner extent may have a predetermined value, and the inner liner 114 may be configured to prevent stretching or expanding radially outward beyond the predetermined value.

In at least some embodiments, the inner liner 114 may be substantially and/or completely compliant, and/or the inner liner 114 may have no radial self-bias. For example, the inner liner 114 may have no radially inward self-bias and/or no radially outward self-bias. In at least some embodiments, the inner liner 114 may be non-self-supporting and may not include a mechanism to radially expand and/or open on its own (e.g., absent a radially outward force exerted upon the inner liner 114 from inside the lumen 120). For example, the inner liner 114 may require a device or object (e.g., a medical device, etc.) that has a greater outer diameter than the first inner diameter and/or the first inner extent of the lumen 120 to push and/or urge the inner liner 114 radially outward from the delivery configuration and/or the folded configuration toward the expanded configuration.

In some embodiments, the inner liner 114 may not require a radially inward force to be applied to the outer surface of the inner liner 114 to collapse radially inward when there is no device, object, etc. disposed within the lumen 120. For example, the inner liner 114 may not be held open or maintain a particular expanded size on its own, or the inner liner 114 may be non-self-supporting. Similarly, the inner liner 114 may not be biased to collapse radially inwardly on its own. For example, the inner liner 114 may take the shape and/or form of, and/or the outer surface of the inner liner 114 may align with and/or be in contact with, surrounding tissue(s) after being expanded from the delivery configuration and/or the folded configuration toward and/or into the expanded configuration. In one example, a constriction in or of a vessel or body lumen in which the inner liner 114 is disposed may urge the inner liner 114 radially inward in the absence of the radially outward force, object, device, etc. that expanded the inner liner 114, but the inner liner 114 is not self-biased radially inwardly on its own (e.g., the inner liner 114 may have zero return force after expanding/opening).

In some embodiments, the inner liner 114 may be formed from a compliant material. In some embodiments, the inner liner 114 may be formed from an inelastic material. In another embodiment, the inner liner 114 may be formed from an elastic material or a combination of inelastic and elastic materials. In some embodiments, the inner liner 114 may be configured to prevent axial stretching of the inner liner 114 along the lumen 120. For example, the inner liner 114 may be configured to expand radially outward from the central longitudinal axis without stretching or expanding in an axial or longitudinal direction along the central longitudinal axis. In some embodiments, the inner liner 114 may be formed from a polymeric and/or plastic material. In some embodiments, the inner liner 114 may be formed from a non-thermoplastic material designed to resist melting while heat is applied to reflow other and/or adjacent components as discussed herein. For example, in at least some embodiments, the inner liner 114 may be formed from a material that has a higher melting point than the outer sheath 112. Some suitable, but non-limiting, examples of materials for the outer sheath 112, the inner liner 114, etc. are discussed below.

In some embodiments, the inner surface of the wall of the inner liner 114 may include one or more layers or coatings, such as but not limited to, a lubricious coating, a hydrophilic coating, a hydrophobic coating, and/or other suitable coatings and the like. In some embodiments, the inner liner 114 may include a lubricant disposed on the inner surface of the wall of the inner liner 114 and/or within the lumen 120. In some embodiments, an outer surface of the outer sheath 112 may include one or more layers or coatings, such as but not limited to, a lubricious coating, a hydrophilic coating, a hydrophobic coating, and/or other suitable coatings and the like. In some embodiments, the outer sheath 112 may include a lubricant disposed on the outer surface of the outer sheath 112.

In some embodiments, the elongate tubular member 110 may comprise one or more reinforcing members extending along its length. In some embodiments, the one or more reinforcing members may include a plurality of longitudinal spines circumferentially spaced around the central longitudinal axis. In some embodiments, the one or more reinforcing members may be fixedly attached to and/or embedded within the outer sheath 112. Other configurations are also contemplated.

In some embodiments, the outer sheath 112 may be fixedly attached to the inner liner 114. In some embodiments, the outer sheath 112 may include at least one perforation 113 (e.g., at least one aperture, at least one window, at least one opening, etc.) formed in and/or extending along at least a portion of a wall of the outer sheath 112 and/or extending along at least a portion of a length of the outer sheath 112.

In some embodiments, the at least one perforation 113 may include and/or define a plurality of elements (e.g., a plurality of apertures, a plurality of windows, a plurality of openings, a plurality of notches, a plurality of holes, a plurality of weakening features, etc.) therein. In some embodiments, the at least one perforation may extend laterally and/or transversely through the wall of the outer sheath 112. In some embodiments, the at least one perforation 113 may effectively remove at least a portion of the wall of the outer sheath 112. In some embodiments, the at least one perforation 113 may only partially extend laterally and/or transversely through the wall of the outer sheath 112, so as to form an area of reduced wall thickness of the outer sheath 112. In some embodiments, the at least one perforation 113 may extend completely through the wall of the outer sheath 112, while in other embodiments, the at least one perforation 113 may be formed as a substantially “thinner” section of the wall of the outer sheath 112. Other configurations, including combinations thereof, are also contemplated. In some embodiments, the outer sheath 112 may extend continuously around the circumference of the inner liner 114. In some embodiments, at least a portion of the outer sheath 112 may be discontinuous.

In some embodiments, the at least one perforation 113 forms a preferred and/or preferential tear line along the outer sheath 112 configured to split apart in response to expansion of the inner liner 114 from the delivery configuration and/or the folded configuration toward the expanded configuration. In some embodiments, the outer sheath 112 may be configured to separate, split, perforate, and/or tear as the inner liner 114 is expanded radially outward from the delivery configuration and/or the folded configuration toward the expanded configuration. In some embodiments, the outer sheath 112 may be configured to separate, split, perforate, and/or tear along and/or through the at least one perforation 113 formed in the wall of the outer sheath 112. In some embodiments, the outer sheath 112 may be configured to separate, split, perforate, and/or tear where the outer sheath 112 is discontinuous and/or constructed of and/or formed from a thinner material.

In at least some embodiments, the at least one perforation 113 may be axially aligned with the central longitudinal axis. In some embodiments, the outer sheath 112 includes at least one perforation 113 disposed over, radially aligned with, and/or circumferentially overlapping each folded portion of the inner liner 114. In some embodiments, the at least one perforation 113 may be arranged in one or more longitudinal and/or axial lines along the length of the outer sheath 112. In some embodiments, the one or more longitudinal and/or axial lines may directly correspond to and/or align with the at least one folded portion.

The outer sheath 112 may include a given wall thickness at various locations along its length. In some embodiments, the wall thickness of the outer sheath 112 may vary along its length. In some embodiments, the wall thickness of the outer sheath 112 may be tapered about the circumference of the inner liner 114 such that a reduced thickness region of the outer sheath 112 is disposed adjacent to, radially outward of, circumferentially aligned with, in communication with, and/or directly over the at least one folded portion. In some embodiments, the reduced thickness region is the at least one perforation 113.

In some embodiments, the at least one perforation may be spaced apart from each other axially, longitudinally, and/or circumferentially, etc. In some embodiments, the at least one perforation 113 proximate a distal end of the outer sheath 112 may have elements thereof spaced apart from each other by a first distance. In some embodiments, the at least one perforation 113 proximate a proximal end of the outer sheath 112 may have elements thereof spaced apart from each other by a second distance, wherein the second distance is greater than the first distance. In some embodiments, the first distance may be greater than the second distance. In some embodiments, the first distance and the second distance may be substantially equal. In some embodiments, spacing between adjacent elements of the at least one perforation may vary along the length of the outer sheath 112. In some embodiments, spacing between adjacent elements may gradually increase, gradually decrease, and/or combinations thereof along the length of the outer sheath 112.

In some embodiments, the at least one perforation 113 may have and/or define a surface area. As used herein, the term “surface area”, with respect to the at least one perforation 113, may be defined as the “area” bounded by the shape, outline, and/or perimeter of one of the at least one perforation 113. In some embodiments, the at least one perforation 113 may have a polygonal shape, a regular shape, an irregular shape, a round shape, a triangular shape, a square or rectangular shape, a hexagonal shape, an octagonal shape, a trapezoidal shape, a diamond shape, or any other suitable shape. For example, in an embodiment where the at least one perforation 113 each has a polygonal shape, the “surface area” of a given element may be defined as the area bounded by an individual polygon. In some embodiments, the surface area of the at least one perforation 113 may be constant and/or equal along the length of the outer sheath 112. In some embodiments, the surface area of the at least one perforation 113 may vary along the length of the outer sheath 112. In some embodiments, the surface area of the at least one perforation 113 may decrease gradually from the distal end of the outer sheath 112 toward the proximal end of the outer sheath 112. In one example, the at least one perforation 113 may include a plurality of windows, and the plurality of windows may decrease in size from the distal end of the outer sheath 112 toward the proximal end of the outer sheath 112. Other configurations, including but not limited to combinations of the features described herein, are also contemplated.

In some embodiments, the elongate tubular member 110 and/or the lumen 120 may be configured to slidably receive a medical device 200. In at least some embodiments, the medical device 200 may be configured to radially expand upon deployment from the lumen 120 and/or the elongate tubular member 110. In some embodiments, the medical device 200 may be self-expanding and/or self-biased toward an expanded configuration. For the purpose of illustration only, the medical device 200 is shown and/or designated in the figures with a dashed oval. In some embodiments, the medical device 200 may be and/or may include a replacement heart valve implant, an occlusive implant, an inflatable balloon, an embolic protection device, or other expandable device(s). In at least some embodiments, an elongate member 210 may extend proximally from the medical device 200 and/or the elongate member 210 may be slidable disposed within elongate tubular member 110 and/or the lumen 120. In some embodiments, the elongate member 210 may be coupled and/or attached to the medical device 200. In some embodiments, the elongate member 210 may be selectively and/or removably coupled and/or attached to the medical device 200.

In some embodiments, the medical device 200 may have a first outer extent in a radially constrained configuration that is greater than the first inner diameter and/or the first inner extent of the elongate tubular member 110 and/or the lumen 120 and/or the inner liner 114 at the distal region and/or the distal end of the elongate tubular member 110. Accordingly, as the medical device 200 is advanced through the lumen 120, the medical device 200 may apply a radially outward force against the inner liner 114 and the inner liner 114 may radially expand from the delivery configuration and/or the folded configuration toward the expanded configuration and/or the outer sheath 112 may separate, split, perforate, and/or tear as the inner liner 114 is expanded radially outward from the delivery configuration and/or the folded configuration toward the expanded configuration, thereby permitting the medical device 200 to pass therethrough in the radially constrained configuration.

Alternatively, in some embodiments, a separate dilator (not shown) may be inserted and/or advanced through the elongate tubular member 110 and/or the lumen 120 before advancing the medical device 200 through the elongate tubular member 110 and/or the lumen 120. The dilator may have an outer extent greater than the first inner diameter and/or the first inner extent of the inner liner 114. The dilator may apply the radially outward force against the inner liner 114 to radially expand the inner liner 114 from the delivery configuration and/or the folded configuration toward the expanded configuration, and/or to cause the outer sheath 112 to separate, split, perforate, and/or tear as the inner liner 114 is expanded radially outward from the delivery configuration and/or the folded configuration toward the expanded configuration. Thereafter, the dilator may be removed from the lumen 120 and the medical device 200 may be inserted into and/or advanced through the lumen 120.

After deploying the medical device 200 from the elongate tubular member 110 and/or the lumen 120 at a treatment site, the medical device 200 may be configured to radially expand when unconstrained by the elongate tubular member 110, as seen in FIG. 1. In some embodiments, deploying the medical device 200 from the elongate tubular member 110 and/or the lumen 120 may comprise advancing the medical device 200 and/or the elongate member 210 through the lumen 120.

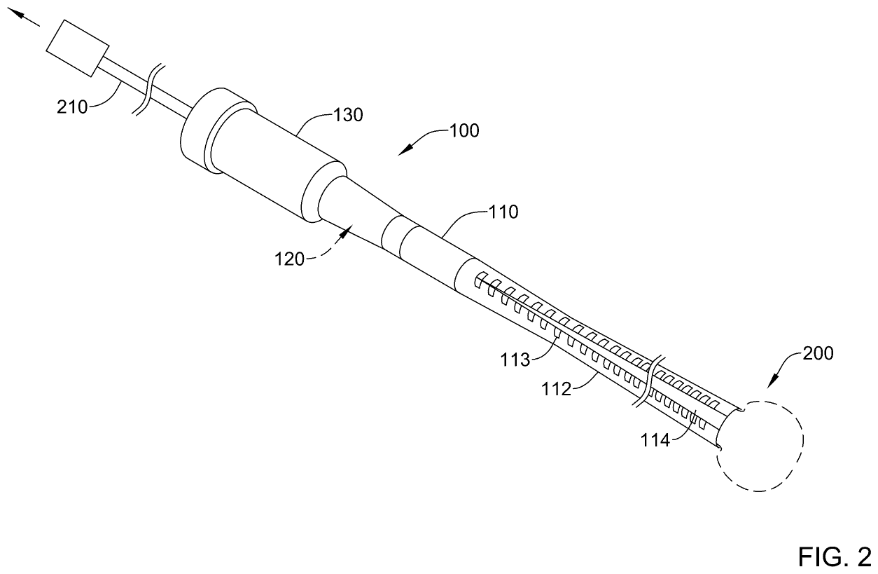

In some embodiments, the medical device 200 may have a second outer extent when unconstrained and/or in an unconstrained configuration that is greater than the first outer extent of the medical device 200 and/or is greater than the second inner diameter and/or the second inner extent of the inner liner 114 and/or the elongate tubular member 110 at the distal region and/or the distal end of the elongate tubular member 110. In some procedures, it may be necessary to recapture, reposition, and/or remove the medical device 200 after initial deployment. However, after expanding the medical device 200 from the radially constrained configuration, recapture from the unconstrained configuration may sometimes be difficult. In some instances, the medical device 200 may not readily collapse and/or may bind up, crumple, or otherwise interfere with the distal end of the elongate tubular member 110, as seen in FIG. 2, thereby preventing the medical device 200 from being recaptured and/or withdrawn into the lumen 120 and/or causing damage to the distal end of the elongate tubular member 110, which may subsequently cause injury to the patient as the elongate tubular member 110 is moved within the patient's anatomy, (e.g., during withdrawal of the elongate tubular member 110, etc.).

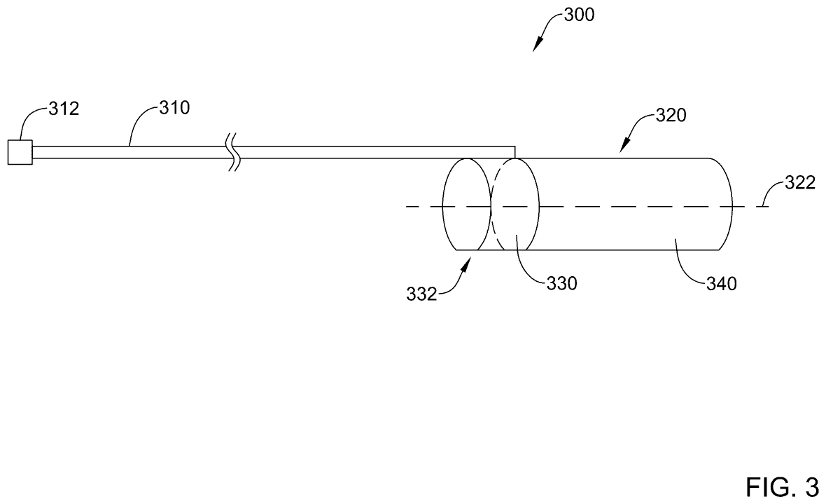

FIG. 3 illustrates selected aspects of a device 300 for aiding in recapturing the medical device 200. The device 300 may comprise an elongate shaft 310 and an expandable sheath extension 320 coupled to a distal end of the elongate shaft 310. In some embodiments, the elongate shaft 310 may comprise a handle member 312 disposed at and/or proximate a proximal end of the elongate shaft 310. For the purpose of illustration, the handle member 312 is shown in the figures as a square block, but the skilled artisan will recognize that other configurations and/or forms are also possible. For example, the handle member 312 could have an elongated shape, a barrel shape, a pistol grip, a knurled surface, or other known handle configurations.

In some embodiments, the expandable sheath extension 320 may be fixedly attached to the distal end of the elongate shaft 310. In some embodiments, the expandable sheath extension 320 extend laterally from and/or may be laterally offset from the elongate shaft 310. In at least some embodiments, the expandable sheath extension 320 may extend distally from and/or distally of the distal end of the elongate shaft 310. In at least some embodiments, the elongate shaft 310 may be formed from a metallic material. In some embodiments, the elongate shaft 310 may be formed from a polymeric material. Some suitable, but non-limiting, examples of materials for the elongate shaft 310 including metallic materials, polymeric materials, etc., and/or combinations thereof, are described below.

In some embodiments, the expandable sheath extension 320 may extend circumferentially around and/or about a central longitudinal axis 322 extending parallel to the elongate shaft 310 and/or a longitudinal axis of the elongate shaft 310, as seen in FIG. 3. In some embodiments, the expandable sheath extension 320 may extend along and/or parallel to the central longitudinal axis 322. In some embodiments, the expandable sheath extension 320 may be circumferentially discontinuous. In some embodiments, the expandable sheath extension 320 may form a tubular structure or a tubular member but is not constructed from a complete tube.

In some embodiments, the expandable sheath extension 320 may comprise a C-shaped collar 330 proximate a proximal end of the expandable sheath extension 320. In some embodiments, the C-shaped collar 330 may be disposed at the proximal end of the expandable sheath extension 320. In some embodiments, the expandable sheath extension 320 may extend distally from the C-shaped collar 330. The C-shaped collar 330 may be fixedly attached to the elongate shaft 310. In some embodiments, the C-shaped collar 330 may be fixedly attached to the distal end of the elongate shaft 310. In some embodiments, the C-shaped collar 330 may be at least partially embedded within the expandable sheath extension 320. In at least some embodiments, the C-shaped collar 330 may be formed from a metallic material. In some embodiments, the C-shaped collar 330 may be formed from a polymeric material. Some suitable, but non-limiting, examples of materials for the C-shaped collar 330 including metallic materials, polymeric materials, etc., and/or combinations thereof, are described below.

In some embodiments, the expandable sheath extension 320 may comprise at least one polymeric sheet 340. In some embodiments, the expandable sheath extension 320 and/or the at least one polymeric sheet 340 may comprise a plurality of polymeric sheets. In some embodiments, the expandable sheath extension 320, the at least one polymeric sheet 340, and/or the plurality of polymeric sheets may comprise two polymeric sheets, three polymeric sheets, four polymeric sheets, etc. In some embodiments, the plurality of polymeric sheets may be laminated together and/or bonded together to form a layered structure. In some embodiments, the plurality of polymeric sheets may be melted, melt bonded, molecularly intermingled, and/or coextruded together to form a single structure. In some embodiments, the plurality of polymeric sheets may be integrally formed and/or monolithically formed with each other. In some embodiments, the plurality of polymeric sheets may extend from a common spine or base element. Other configurations are also contemplated.

In some embodiments, the at least one polymeric sheet 340 may be rolled into a tubular member. In some embodiments, the expandable sheath extension 320 may be self-biased toward and/or to a radially collapsed configuration (e.g., FIG. 3). In some embodiments, the at least one polymeric sheet 340 may be rolled into the tubular member in the radially collapsed configuration. In some embodiments, the expandable sheath extension 320 may circumferentially overlap itself to form the tubular member in the radially collapsed configuration. In some embodiments, the at least one polymeric sheet 340 and/or the plurality of polymeric sheets may circumferentially overlap each other to form the tubular member in the radially collapsed configuration.

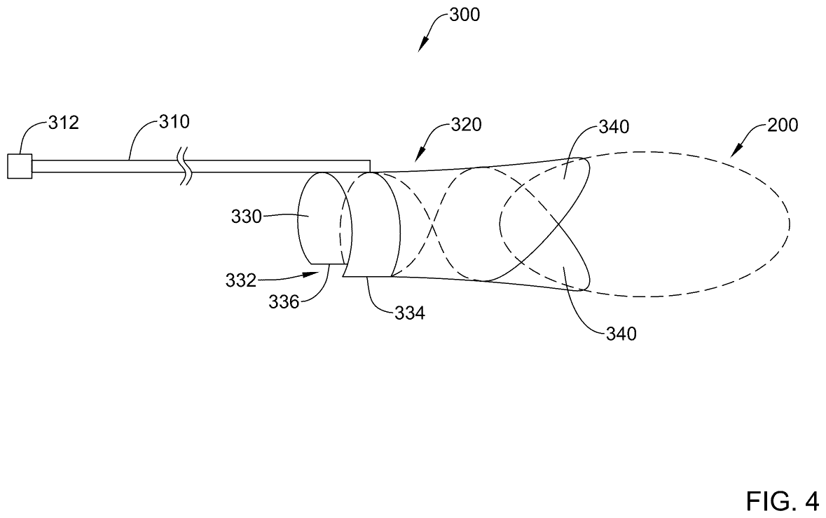

In some embodiments, the expandable sheath extension 320 and/or the at least one polymeric sheet 340 may be configured to expand radially and/or circumferentially from the radially collapsed configuration upon application of a radially outward force against an inner surface of the expandable sheath extension 320, as seen in FIG. 4. In some embodiments, application of the radially outward force against the inner surface of the expandable sheath extension 320 causes the expandable sheath extension 320 and/or the at least one polymeric sheet 340 to expand radially and/or circumferentially.

In some embodiments, the C-shaped collar 330 may be at least partially embedded within the at least one polymeric sheet 340. The C-shaped collar 330 is circumferentially discontinuous. In some embodiments, the C-shaped collar 330 may comprise a gap 332 defined by a first end 334 of the C-shaped collar 330 and a second end 336 of the C-shaped collar 330 disposed opposite the first end 334. In some embodiments, the gap 332 may be positioned generally opposite the elongate shaft 310. In some embodiments, the C-shaped collar 330 does not circumferentially overlap itself in any configuration. In some alternative embodiments, the C-shaped collar 330 may circumferentially overlap itself in the radially collapsed configuration and may not circumferentially overlap itself when radially and/or circumferentially expanded from the radially collapsed configuration.

In some embodiments, the expandable sheath extension 320 and/or the tubular member may have a first outer diameter in the radially collapsed configuration and a second outer diameter greater than the first outer diameter when radially and/or circumferentially expanded. In some embodiments, the first outer diameter of the expandable sheath extension 320 and/or the tubular member may be less than 6.000 millimeters (0.236 inches). In some embodiments, the first outer diameter of the expandable sheath extension 320 and/or the tubular member may be less than 5.667 millimeters (0.223 inches). In some embodiments, the first outer diameter of the expandable sheath extension 320 and/or the tubular member may be less than 5.333 millimeters (0.210 inches). In some embodiments, the first outer diameter of the expandable sheath extension 320 and/or the tubular member may be less than 5.000 millimeters (0.197 inches). In some embodiments, the first outer diameter of the expandable sheath extension 320 and/or the tubular member may be less than 4.667 millimeters (0.184 inches). In some embodiments, the first outer diameter of the expandable sheath extension 320 and/or the tubular member may be less than 4.333 millimeters (0.170 inches). In some embodiments, the first outer diameter of the expandable sheath extension 320 and/or the tubular member may be less than 4.000 millimeters (0.158 inches). In some embodiments, the first outer diameter of the expandable sheath extension 320 and/or the tubular member may be less than 3.667 millimeters (0.144 inches). In some embodiments, the first outer diameter of the expandable sheath extension 320 and/or the tubular member may be less than 3.333 millimeters (0.131 inches). In some embodiments, the first outer diameter of the expandable sheath extension 320 and/or the tubular member may be less than 3.000 millimeters (0.118 inches). Other configurations are also contemplated.

In some embodiments, the first outer diameter of the expandable sheath extension 320 and/or the tubular member may be less than the first inner diameter and/or the first inner extent of the elongate tubular member 110 and/or the lumen 120 and/or the inner liner 114 at the distal region and/or the distal end of the elongate tubular member 110. In some embodiments, the first outer diameter of the expandable sheath extension 320 and/or the tubular member may be less than the second inner diameter and/or the second inner extent of the elongate tubular member 110 and/or the lumen 120 and/or the inner liner 114 at the distal region and/or the distal end of the elongate tubular member 110.

In some embodiments, the expandable sheath extension 320 and/or the tubular member may have a first inner diameter in the radially collapsed configuration and a second inner diameter greater than the first inner diameter when radially and/or circumferentially expanded. In some embodiments, the first inner diameter of the expandable sheath extension 320 and/or the tubular member may be at least 1.000 millimeters (0.039 inches). In some embodiments, the first inner diameter of the expandable sheath extension 320 and/or the tubular member may be at least 1.333 millimeters (0.053 inches). In some embodiments, the first inner diameter of the expandable sheath extension 320 and/or the tubular member may be at least 1.667 millimeters (0.066 inches). In some embodiments, the first inner diameter of the expandable sheath extension 320 and/or the tubular member may be at least 2.000 millimeters (0.079 inches). In some embodiments, the first inner diameter of the expandable sheath extension 320 and/or the tubular member may be at least 2.333 millimeters (0.092 inches). In some embodiments, the first inner diameter of the expandable sheath extension 320 and/or the tubular member may be at least 2.667 millimeters (0.105 inches). Other configurations are also contemplated. In some embodiments, the first inner diameter of the expandable sheath extension 320 and/or the tubular member may be greater than an outer diameter of the elongate member 210.

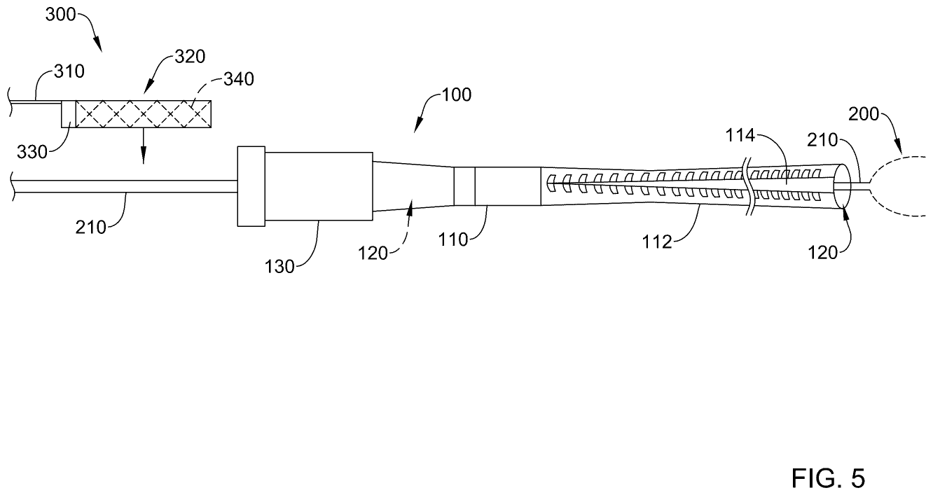

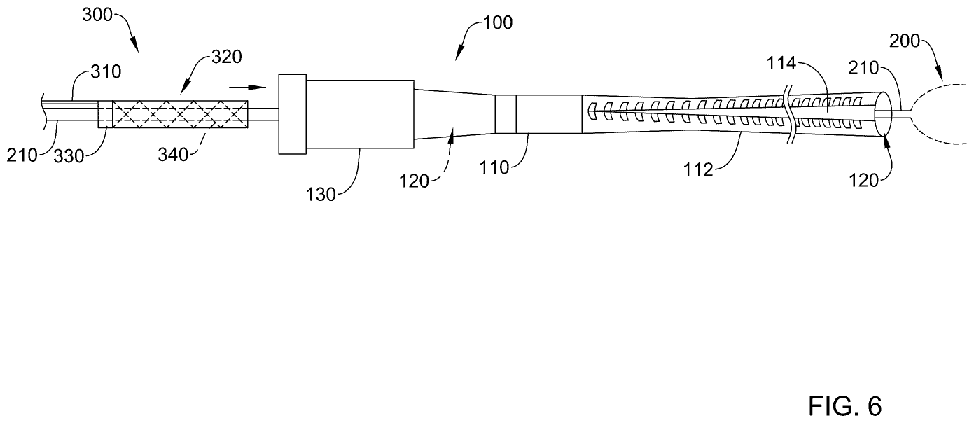

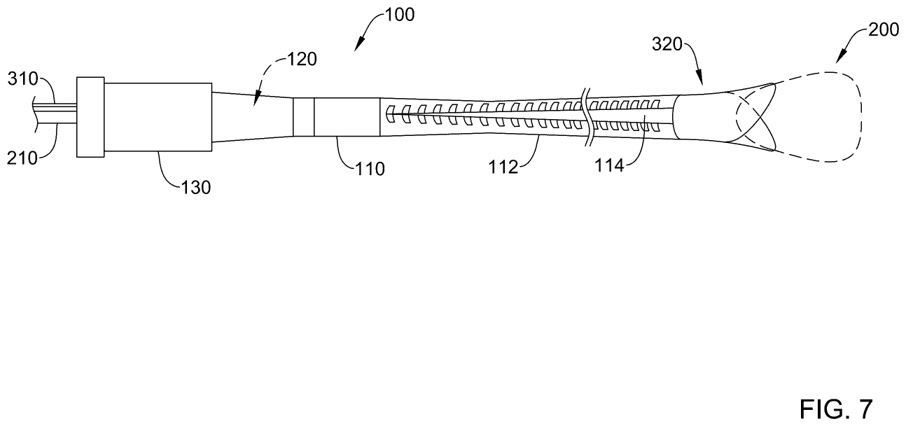

A system for deploying and recapturing a medical device 200 may comprise the deployment catheter 100 described herein and the device 300 for aiding in recapturing the medical device 200 after the medical device 200 has been deployed distal of the elongate tubular member 110 of the deployment catheter 100, as seen in FIGS. 5-6. In some embodiments, the elongate shaft 310 of the device 300 may be configured to slidably advance the expandable sheath extension 320 through the inner liner 114 and/or the lumen 120 of the elongate tubular member 110 and/or the deployment catheter 100. In some embodiments, the elongate shaft 310 of the device 300 may be configured to slidably advance the expandable sheath extension 320 through the lumen 120 of the elongate tubular member 110 and/or the deployment catheter 100 to a guiding position, shown in FIG. 7. In some embodiments, the elongate shaft 310 of the device 300 may be sized, shaped, and/or configured to slide longitudinally and/or axially alongside the elongate member 210 within the lumen 120 of the elongate tubular member 110 and/or the inner liner 114.

In some embodiments, in the guiding position, a proximal end of the expandable sheath extension 320 may be disposed within the lumen 120 of the elongate tubular member 110 and/or within the inner liner 114 and a distal end of the expandable sheath extension 320 may be disposed distal of a distal end of the elongate tubular member 110. The expandable sheath extension 320 may be advanced through the lumen 120 of the elongate tubular member 110 and/or the deployment catheter 100 to the guiding position in the radially collapsed configuration. Upon engagement of the medical device 200 with the expandable sheath extension 320 and/or upon application of the radially outward force against the inner surface of the expandable sheath extension 320, by the medical device 200 for example, the expandable sheath extension 320 may expand radially and/or circumferentially. In particular, the distal end of the expandable sheath extension 320 may expand radially and/or circumferentially while the proximal end of the expandable sheath extension 320 is constrained in and/or near the radially collapsed configuration by the distal end of the elongate tubular member 110 and/or the inner liner 114, thereby causing the expandable sheath extension 320 to assume a generally conical shape opening and/or radially expanding in a distal direction. The expandable sheath extension 320 may form a funnel and/or a tapered structure to compress the medical device 200 radially inward as the medical device 200 is pulled and/or guided into the lumen 120 and/or the inner liner 114. As such, in the guiding position, the expandable sheath extension 320 may be configured to guide the medical device 200 radially inward toward the lumen 120 of the elongate tubular member 110 and/or the inner liner 114 as the medical device 200 is pulled proximally.

FIGS. 5-8 schematically illustrate selected aspects of a method of deploying and recapturing the medical device 200. In some embodiments, the method may comprise deploying the medical device 200 from the deployment catheter 100 comprising the elongate tubular member 110 having the lumen 120 extending therethrough, as seen in FIG. 5. In some embodiments, the method may comprise pushing and/or advancing the medical device 200 through and/or out of the elongate tubular member 110, the lumen 120, and/or the inner liner 114 with the elongate member 210 extending proximally from the medical device 200. In some embodiments, the medical device 200 may be disposed at the distal end of the elongate member 210.

In some embodiments, after deploying the medical device 200 from the deployment catheter 100, the elongate tubular member 110, the lumen 120, and/or the inner liner 114, the medical device 200 may radially expand to an outer diameter and/or an outer extent greater than an inner diameter and/or an inner extent of the elongate tubular member 110, the lumen 120, and/or the inner liner 114 at the distal end of the elongate tubular member 110. In some embodiments, after deploying the medical device 200 from the deployment catheter 100, the elongate tubular member 110, the lumen 120, and/or the inner liner 114, the medical device 200 may radially expand to the second outer diameter and/or the second outer extent greater than the second inner diameter and/or the second inner extent of the elongate tubular member 110, the lumen 120, and/or the inner liner 114 at the distal end of the elongate tubular member 110.

In some embodiments, the method may comprise positioning the expandable sheath extension 320 of the device 300 coupled to the distal end of the elongate shaft 310 of the device 300 in a first position alongside the elongate member 210, as seen in FIG. 5. The expandable sheath extension 320 may extend laterally from and/or may be laterally offset from the elongate shaft 310. In at least some embodiments, the expandable sheath extension 320 may be self-biased toward the radially collapsed configuration.

In some embodiments, the expandable sheath extension 320 may be configured to be translated in a lateral direction relative to the elongate member 210 extending proximally from the medical device 200 through the lumen 120 of the elongate tubular member 110 to shift the expandable sheath extension from a first position alongside the elongate member 210 to a second position wherein the elongate member 210 extends longitudinally and/or axially through the expandable sheath extension 320 and/or wherein the expandable sheath extension 320 surrounds the elongate member 210.

In some embodiments, the method may comprise translating the device 300 and/or the expandable sheath extension 320 from the first position in a lateral direction relative to and/or toward the elongate member 210 to a second position wherein the elongate member 210 extends longitudinally and/or axially through the expandable sheath extension 320, as seen in FIGS. 5-6. In some embodiments, the method may comprise translating the device 300 and/or the expandable sheath extension 320 from the first position in the lateral direction relative to and/or toward the elongate member 210 to the second position wherein the expandable sheath extension 320 surrounds the elongate member 210. In some embodiments, when the expandable sheath extension 320 is disposed in the second position in the radially collapsed configuration, at least a portion of the expandable sheath extension 320 completely surrounds the elongate member 210. In some embodiments, when the expandable sheath extension 320 is disposed in the second position in the radially collapsed configuration, at least a portion of the tubular member completely surrounds the elongate member 210. In some embodiments, when the expandable sheath extension 320 is disposed in the second position in the radially collapsed configuration, the at least one polymeric sheet 340 and/or the plurality of polymeric sheets completely surrounds the elongate member 210.

In some embodiments, the method may comprise advancing the device 300 and/or the expandable sheath extension 320 along the elongate member 210 to the guiding position proximate the distal end of the elongate tubular member 110, as seen in FIGS. 6-7. In some embodiments, the method may comprise advancing the device 300 and/or the expandable sheath extension 320 within the elongate tubular member 110, the lumen 120, and/or the inner liner 114 to the guiding position proximate the distal end of the elongate tubular member 110. In some embodiments, the method may comprise advancing the device 300 and/or the expandable sheath extension 320 through the elongate tubular member 110, the lumen 120, and/or the inner liner 114 to the guiding position proximate the distal end of the elongate tubular member 110.

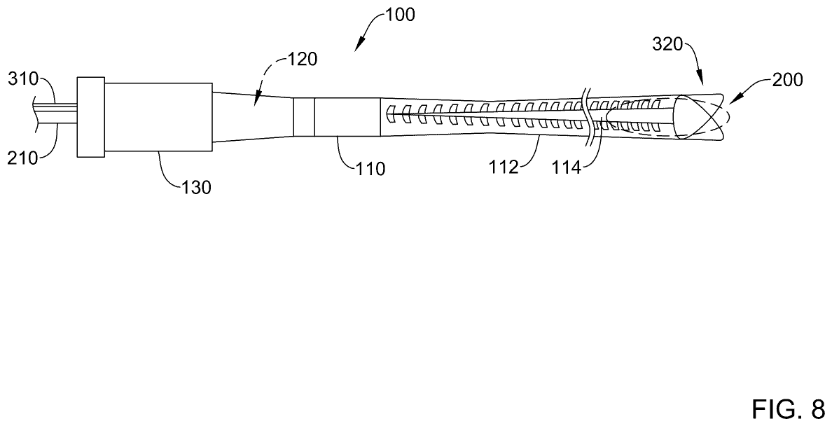

In some embodiments, the method may comprise pulling the medical device 200 proximally into the elongate tubular member 110, the lumen 120, and/or the inner liner 114 through the expandable sheath extension 320 of the device 300, as seen in FIG. 8. In some embodiments, the expandable sheath extension 320 may be configured to expand radially and/or circumferentially upon engagement with the medical device 200. In some embodiments, the expandable sheath extension 320 may be configured to expand radially and/or circumferentially when a radially outward force is applied to an inner surface of the expandable sheath extension 320. In some embodiments, the expandable sheath extension 320 may be configured to expand radially and/or circumferentially as and/or while the medical device 200 is being pulled into and/or through the expandable sheath extension 320.

In some embodiments, the method may comprise withdrawing the expandable sheath extension 320 into the elongate tubular member 110, the lumen 120, and/or the inner liner 114. In some embodiments, the method may comprise withdrawing the expandable sheath extension 320 into the elongate tubular member 110, the lumen 120, and/or the inner liner 114 along with and/or alongside the medical device 200. In some embodiments, the method may comprise withdrawing the expandable sheath extension 320 into the elongate tubular member 110, the lumen 120, and/or the inner liner 114 after the medical device 200.

In some embodiments, the method may comprise withdrawing the deployment catheter 100 from the patient's anatomy. In some embodiments, the method may comprise withdrawing the medical device 200 completely through the deployment catheter 100, the elongate tubular member 110, the lumen 120, and/or the inner liner 114. In some embodiments, the method may comprise withdrawing the medical device 200 completely through the deployment catheter 100, the elongate tubular member 110, the lumen 120, and/or the inner liner 114 before withdrawing the deployment catheter 100 from the patient's anatomy. Other configurations are also contemplated.

The materials that can be used for the various components of the devices, systems, and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the elongate shaft, the expandable sheath extension, the deployment catheter, the elongate tubular member, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly (alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-clastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate. the use of any of the features of one example embodiment being used in other embodiments. The scope of the disclosure is, of course, defined in the language in which the appended claims are expressed.

What is claimed is:

1. A device for aiding in recapturing a medical device, comprising:
an elongate shaft; and
an expandable sheath extension coupled to a distal end of the elongate shaft;
wherein the expandable sheath extension is laterally offset from the elongate shaft;
wherein the expandable sheath extension is self-biased toward a radially collapsed configuration.

2. The device of claim 1, wherein the expandable sheath extension extends circumferentially around a central longitudinal axis extending parallel to the elongate shaft;
wherein the expandable sheath extension is circumferentially discontinuous.

3. The device of claim 1, wherein the expandable sheath extension comprises a C-shaped collar proximate a proximal end of the expandable sheath extension.

4. The device of claim 3, wherein the C-shaped collar is fixedly attached to the elongate shaft.

5. The device of claim 3, wherein the C-shaped collar is formed from a metallic material.

6. The device of claim 3, wherein the expandable sheath extension comprises at least one polymeric sheet rolled into a tubular member in the radially collapsed configuration.

7. The device of claim 6, wherein the C-shaped collar is at least partially embedded within the at least one polymeric sheet.

8. The device of claim 6, wherein the tubular member has an outer diameter of less than 4.667 millimeters in the radially collapsed configuration.

9. The device of claim 6, wherein application of a radially outward force against an inner surface of the expandable sheath extension causes the at least one polymeric sheet to expand radially and circumferentially.

10. A system for deploying and recapturing a medical device, comprising:
a deployment catheter comprising an elongate tubular member having a lumen extending therethrough, the lumen being configured to slidably receive a medical device; and
a device for aiding in recapturing the medical device after the medical device has been deployed distal of the elongate tubular member, the device comprising:
an elongate shaft; and
an expandable sheath extension coupled to a distal end of the elongate shaft;
wherein the expandable sheath extension is laterally offset from the elongate shaft;
wherein the expandable sheath extension is self-biased toward a radially collapsed configuration;
wherein the elongate shaft is configured to slidably advance the expandable sheath extension through the lumen of the elongate tubular member to a guiding position wherein a proximal end of the expandable sheath extension is disposed within the lumen of the elongate tubular member and a distal end of the expandable sheath extension is disposed distal of the elongate tubular member.

11. The system of claim 10, wherein in the guiding position the expandable sheath extension is configured to guide the medical device radially inward toward the lumen of the elongate tubular member as the medical device is pulled proximally.

12. The system of claim 10, wherein the medical device has an outer extent in an unconstrained configuration that is greater than an inner diameter of the elongate tubular member at a distal end of the elongate tubular member.

13. The system of claim 10, wherein the expandable sheath extension is configured to be translated in a lateral direction relative to an elongate member extending proximally from the medical device through the lumen of the elongate tubular member to shift the expandable sheath extension from a first position alongside the elongate member to a second position the elongate member extends longitudinally through the expandable sheath extension.

14. The system of claim 13, wherein the elongate shaft is sized and configured to slide longitudinally alongside the elongate member within the lumen of the elongate tubular member.

15. A method of deploying and recapturing a medical device, comprising:
deploying a medical device from a deployment catheter, the deployment catheter comprising an elongate tubular member having a lumen extending therethrough, wherein the lumen is configured to slidably receive the medical device disposed at a distal end of an elongate member;
positioning an expandable sheath extension coupled to a distal end of an elongate shaft in a first position alongside the elongate member, wherein the expandable sheath extension is laterally offset from the elongate shaft and wherein the expandable sheath extension is self-biased toward a radially collapsed configuration;
translating the expandable sheath extension from the first position in a lateral direction relative to the elongate member to a second position wherein the elongate member extends longitudinally through the expandable sheath extension;
advancing the expandable sheath extension along the elongate member to a guiding position proximate a distal end of the elongate tubular member; and
pulling the medical device proximally into the elongate tubular member through the expandable sheath extension.

16. The method of claim 15, wherein at least a portion of the expandable sheath extension is configured to expand radially upon engagement with the medical device.

17. The method of claim 15, wherein when the expandable sheath extension is disposed in the second position in the radially collapsed configuration, at least a portion of the expandable sheath extension completely surrounds the elongate tubular member.

18. The method of claim 15, wherein after deploying the medical device from the deployment catheter, the medical device radially expands to an outer extent greater than an inner diameter of the elongate tubular member at a distal end of the elongate tubular member.

19. The method of claim 15, wherein the expandable sheath extension is circumferentially discontinuous.

20. The method of claim 19, wherein the expandable sheath extension circumferentially overlaps itself to form a tubular member in the radially collapsed configuration
